(19) United States
(12) Patent Application Publication
(10) Pub. No.: US 2003/0187331 A1
(43) Pub. Date: Oct. 2, 2003

(54) Ergonomic otoscope with efficient light

(76) Inventors: Jeremy Faludi, Stanford, CA (US); Brian Rulifson, Stanford, CA (US); Jody Cimbalo, Mountain View, CA (US)

Correspondence Address: Carey deRafael, Stanford Office of Technology Licensing, 1705 El Camino Real, Palo Alto, CA, 94306-1106, US

(21) Appl. No.: 10/371,974

(22) Filed: Feb. 20, 2003

Related U.S. Application Data
(60) Provisional application No. 60/359,188, filed on Feb. 22, 2002.

(51) Int. Cl. A61B 1/267

(52) U.S. Cl. 600/200

(57) ABSTRACT

An otoscope with a body designed for improved ergonomics and control, by eliminating the handle and having a shape which accommodates many different-sized hands, as well as letting the practitioner rest most of their hand on the patient's head while using the device. The body shape and color are also less intimidating than existing otoscopes, for a more child-friendly device. The otoscope uses light-emitting diodes for its illumination source. The light from these LED's can be directed into the ear canal either by means of a beam splitter, a prism ring, or a reflective-walled channel.

### CROSS-REFERENCE TO RELATED APPLICATIONS

This application is cross-referenced to and claims priority from U.S. Provisional application 60/359,188 filed Feb. 22, 2002, which is hereby incorporated by reference.

### FIELD OF THE INVENTION

The present invention relates generally to otoscopes. More particularly, the present invention relates to otoscopes with insufflation attachments or integrated insufflators.

### BACKGROUND

Otoscopes are instruments used by doctors or other medical practitioners to look inside a patient's ear and observe the eardrum and/or ear canal. “Insufflation” is the act of puffing air into a body cavity such as the ear canal; this is done for diagnostic purposes, to observe whether the eardrum deflects under pressure or whether fluid behind the eardrum prevents it from deflecting.

A typical otoscope includes a magnifying lens, a tip onto which disposable specula can be mounted, a light and light-routing device, batteries to power the light, and an on/off switch for the light. Their bodies are typically formed of a head portion and a handle portion. Otoscopes generally do not currently perform insulation themselves, but have insufflation attachments composed of a rubber bulb connected to the otoscope head via a plastic tube. The practitioner squeezes the rubber bulb to puff air through the otoscope head into the patient's ear.

Existing otoscopes require an awkward hand position, are heavy and unbalanced, are not conducive to good ergonomics for the practitioner or good control of the patient, and their insufflation attachments often slip out of the practitioner's hand. They are also often perceived as frightening-looking by children.

U.S. Pat. No. 5,873,819 pertains to an otoscope head with integrated insufflator, but it does not solve the ergonomics and control problems which motivated this invention.

The light source typically used for existing otoscopes is an incandescent or halogen bulb. This lighting method is problematic in that it creates large amounts of heat, so it cannot be mounted in the head of the otoscope, as it would cause irritation to the practitioner and/or the patient, and may cause rubber or plastic parts to decay prematurely. Existing otoscopes therefore place the light source in the handle with a metal casing (which becomes uncomfortably hot after prolonged use of the lighting) and use fiberoptics and prisms to route the light through the forward hole in the otoscope head, through the hole in the speculum, into the patient's ear. Current light sources also require large heavy batteries, which are also housed in the otoscope's handle, unless the otoscope is connected by a cord to a power source in the wall. The heavy batteries in the handle cause the otoscope to be awkward and unbalanced, which causes poor ergonomics. They also make the otoscope less portable.

It is the object of the present invention to overcome the deficiencies of prior art.

### SUMMARY OF THE INVENTION

The invention disclosed here, an improved otoscope, has a large body with no projecting handle. An advantage of the present invention over the prior art is that its body is ergonomically shaped, providing improved contact between the practitioner's hand and the patient's head. Thus the practitioner has better control and is less susceptible to patient movement during examination procedures. The shape allows the present otoscope to be steadily, comfortably gripped and manipulated by different-sized hands. In operation, a practitioner holds the otoscope with the thumb and forefinger, and perhaps also the ring finger. The full ulnar surface of the hand, and much of the palmar surface, are in contact with the patient's head, for improved stability and control. The practitioner's middle finger can be used to squeeze the insufflation bulb, or it and the ring finger can be positioned on the patient's head for greater stability.

The present otoscope body has a built-in insufflation bulb, and the body is airtight so that all air displaced by squeezing the insufflation bulb puffs into the patient's ear. The insufflation bulb covers only part of the circumference of the body, it is not a toroid that fully encircles the body. This use of a bulb rather than a toroid improves the practitioner's ability to hold the otoscope without wobbling during insufflation, which is necessary for optimal viewing of the patient's ear canal.

An advantage of the present invention that it reduces manufacturing cost, maintenance cost, weight, and energy use. Thus the otoscope includes light-emitting diodes (LED's) as its light source. These lights use less power than existing light sources, so they create little heat and do not require heavy batteries. The LED's lack of heat generation allows them to be housed in plastic or rubber rather than requiring metal to dissipate and withstand heat generated by the light source. This reduced battery usage and lack of heat generation eliminate the need for a handle or metal casing. Thus, an advantage of the present invention is that manufacturing can be done with very few parts, and with inexpensive materials and methods such as injection-molded plastic or rubber. Another advantage of the present invention is that because the lights can be housed in the head of the otoscope, the light routing method is not restricted to fiberoptics or prisms. The light can be directed into the ear by means of a prism ring as found in the tip of existing otoscopes, or a beam-splitter, or a reflective-walled channel. The lack of fiberoptics will further reduce manufacturing cost. The LED's also have extremely long lifetimes, so they should never require replacement by the user as light sources in existing otoscopes do.

Another advantage is that the present invention allows the otoscope's shape, color, and casing material to be less intimidating-looking than existing otoscopes, for a more child-friendly device.

### BRIEF DESCRIPTION OF THE FIGURES

The objectives and advantages of the present invention will be understood by reading the following detailed description in conjunction with the drawings, in which:

FIG. 1 is the otoscope alone, in perspective view;

FIG. 2 is the otoscope being held by a user; and

FIG. 3 is a cutaway view of possible light routing methods.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

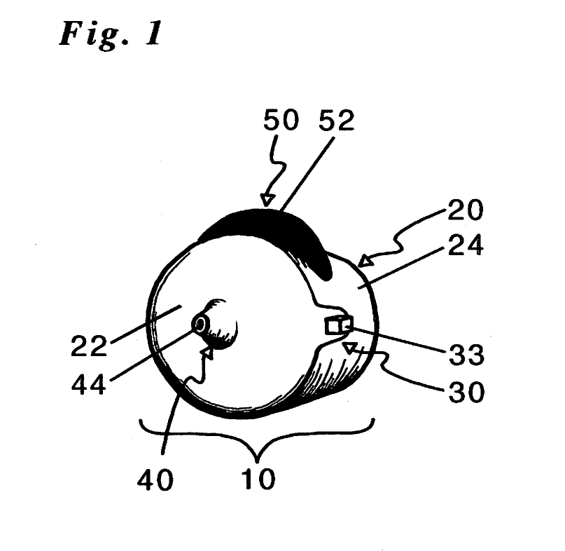

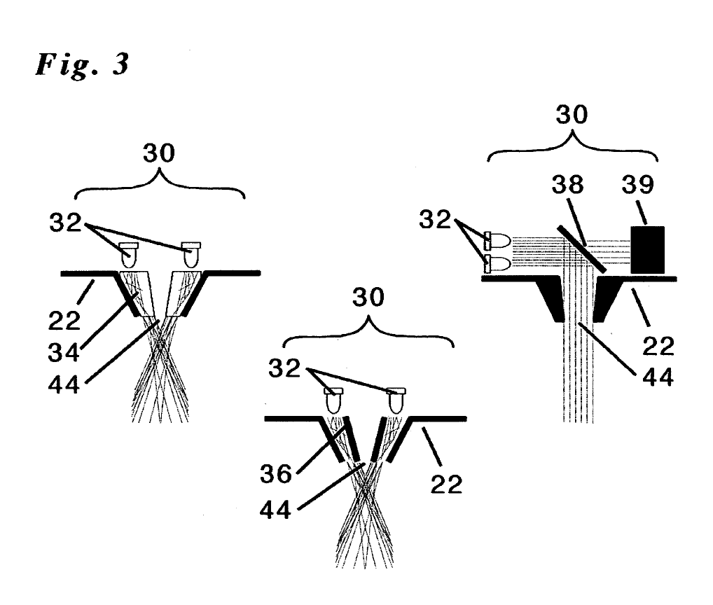

The present invention, shown in FIGS. 1, 2, and 3, provides an otoscope 10 according to the present invention is shown, including a body 20, an illumination system 30, an optical system 40, and an insulation system 50. Not all parts of all systems are visible in each drawing, but system numbers are listed with appropriate assemblies.

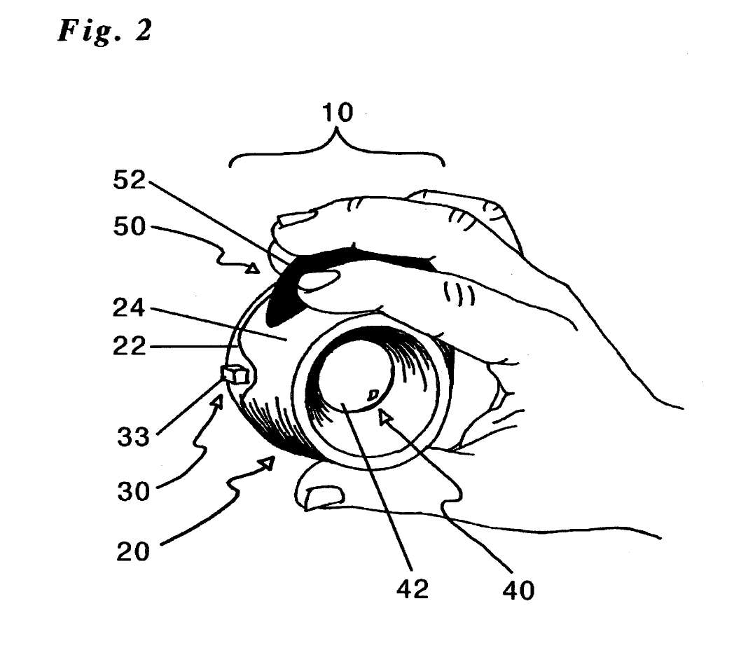

Referring to FIGS. 1 and 2, the body 20 includes a front section 22 and a rear section 24. The body 20 has an ergonomic shape that is unitary, without projections such as a handle. It can be ellipsoid as pictured in FIGS. 1 and 2, but can also take many other forms appropriate to the ergonomics of the hand of a practitioner. It is gripped similarly to how one would grip a football, not how one would grip a pencil or a hammer. The full ulnar surface of the hand, and much of the palmar surface, are in contact with the patient's head during use. The practitioner's ring finger and possibly middle finger can also be positioned on the patient's head during use for greater stability. It is a more ergonomic shape than existing otoscopes, easily gripped by different-sized hands. This shape facilitates better practitioner hand position on the patient's head, so that the practitioner has better control and is less susceptible to patient movement. The ergonomic body holds all components of the otoscope: an illumination system 30 including light(s) and batteries and a switch to turn the illumination on or off; an optical system 40 to clearly magnify an image of the patient's ear canal or tympanic membrane for viewing by the practitioner. The body may also house an insufflation mechanism 50. The front section 22 is built to be compatible with an existing disposable speculum, for patient hygiene. All other systems 30, 40, 50 are contained within or attached to body 20.

Referring to FIGS. 1, 2, and 3, illumination system 30 includes light emitting diodes (LED's) 32 to generate the light. The switch 33 for turning the light on and off is also visible on the side of the body 20. It also includes one of three different methods to route the light through the forward opening 42 of the body 20, into the patient's ear, so that the practitioner has a well-lit view of the patient's ear canal and tympanic membrane. One light routing method uses a circular prism 34, in which total internal reflection inside the prism 34 directs the light appropriately; this is used in most prior art. Another method uses a circular reflective channel 36, where mirrored coatings on the interior walls of a cavity route the light into the patient's ear. In the third method, light shines against a beam-splitter 38 so that part of the light reflects into the ear canal; the light reflected from inside the ear passes through the same beam-splitter 38, and some of it reaches the viewer's eye. In this method, the light that is not used must be directed into a beam-dump 39 such as a black wedge-shaped surface, so that light that shines through the beam splitter does not get reflected from the inside of the otoscope and reflect back off the beam splitter into the practitioner's eye.

Referring to FIGS. 1 and 2, optical system 40 includes an eyepiece 42, mounted in body 20, with a clear line of sight through the forward opening 44 and into the ear canal of the patient so that the practitioner can view a magnified image of the patient's ear canal and eardrum. Optical system 40 can contain additional optics as understood by those skilled in the art, depending on the magnification and field of view desired.

Referring to FIGS. 1 and 2, the insufflation system 50 includes an insufflation mechanism that is integral with the body 20, as in some prior art. A preferred embodiment of the insufflator system 50 is to be an insulator bulb 52. This insufflator bulb 52 can be manufactured as part of the same piece as the rear body 24 but with a thinner wall cross-section, or a material of different durometer. The piece could be cast in flexible material such as plastic or rubber, with the walls of the rear body 24 thick enough to be fairly rigid so that the otoscope can be gripped steadily, while the walls of the insufflation bulb 52 are thin enough to be flexible so that the practitioner can squeeze the bulb with a finger to puff air through the otoscope body into the patient's ear canal. Dual-durometer casting could also be used to provide a flexible insufflator and a rigid body.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed is:

1. A medical device for examining a patient, comprising:
a) a body wherein said body is shaped ergonomically to fit a user's hand, and wherein said body comprises a first opening and a second opening; and
b) a plurality of light sources, for generating a plurality of light beams, located in said body; and
c) an optical path for guiding said plurality of light beams through said second opening.

2. The medical device as set forth in claim 1, wherein said plurality of light sources comprises Light Emitting Diodes (LED's).

3. The medical device as set forth in claim 1, wherein said body comprises injection molded or cast materials.

4. The medical device as set forth in claim 1, wherein said first opening comprises a lens.

5. The medical device as set forth in claim 1, wherein said body comprises a means to select one or more of said plurality of light sources.

6. The medical device as set forth in claim 1, wherein said means to select light sources comprises a switch.

7. The medical device as set forth in claim 1, wherein said means to select light sources comprises means to select the intensity of light.

8. The medical device as set forth in claim 1, wherein said body comprises a means to pass said light through a filter, aperture, or lens.

9. The medical device as set forth in claim 1, wherein said body comprises an insufflating means to insufflate a part of said patient.

10. The medical device as set forth in claim 1, wherein said optical path comprises a beam splitter, a prism, or a cavity with mirrored walls.

11. The medical device as set forth in claim 1, further comprising a speculum.

12. A device for examining a structure or system, comprising:
a) a body wherein said body is shaped ergonomically to fit a user's hand, and wherein said body comprises a first opening and a second opening; and
b) a plurality of light sources, for generating a plurality of light beams, located in said body; and
c) an optical path for guiding said plurality of light beams through said second opening.

13. The device as set forth in claim 12, wherein said plurality of light sources comprises Light Emitting Diodes (LED's).

14. The device as set forth in claim 12, wherein said body comprises injection molded or cast materials.

15. The device as set forth in claim 12, wherein said first opening comprises a lens.

16. The device as set forth in claim 12, wherein said body comprises a means to select one or more of said plurality of light sources.

17. The device as set forth in claim 12, wherein said means to select light sources comprises a switch.

18. The device as set forth in claim 12, wherein said means to select light sources comprises means to select the intensity of light.

19. The device as set forth in claim 12, wherein said body comprises a means to pass said light through a filter, aperture, or lens.

20. The device as set forth in claim 12, wherein said body comprises an insufflating means to insufflate a part of said structure or system.

21. The device as set forth in claim 12, wherein said optical path comprises a beam splitter, a prism, or a cavity with mirrored walls.

22. The device as set forth in claim 12, further comprising a speculum.

23. A method for examining a patient, comprising the steps of:
a) providing a body wherein said body is shaped ergonomically to fit a user's hand, and wherein said body comprises a first opening and a second opening; and
b) providing a plurality of light sources, for generating a plurality of light beams, located in said body; and
c) providing an optical path for guiding said plurality of light beams through said second opening.

24. The method as set forth in claim 23, wherein said plurality of light sources comprises Light Emitting Diodes (LED's).

25. The method as set forth in claim 23, wherein said body comprises injection molded or cast materials.

26. The method as set forth in claim 23, wherein said first opening comprises a lens.

27. The method as set forth in claim 23, wherein said body comprises the step of providing a means to select one or more of said plurality of light sources.

28. The method as set forth in claim 23, wherein said means to select light sources comprises the step of providing a switch.

29. The method as set forth in claim 23, wherein said means to select light sources comprises the step of providing means to select the intensity of light.

30. The method as set forth in claim 23, wherein said body comprises a means to pass said light through a filter, aperture, or lens.

31. The method as set forth in claim 23, wherein said body comprises the step of providing an insufflating means to insufflate a part of said patient.

32. The method as set forth in claim 23, wherein said optical path comprises a beam splitter, a prism, or a cavity with mirrored walls.

33. The method as set forth in claim 23, further comprising the step of providing a speculum.
